(19) United States
(12) Patent Application Publication
(10) Pub. No.: US 2024/0115644 A1
(43) Pub. Date: Apr. 11, 2024

(54) COMPOSITION FOR PREVENTING, IMPROVING OR TREATING GASTRITIS OR PEPTIC ULCER COMPRISING EXTRACT OF CINNAMOMUM CASSIA, FRACTION OF SAID EXTRACT, ISOLATE OF SAID FRACTION OR COMPOUNDS ISOLATED THEREFROM

(71) Applicant: CHONG KUN DANG PHARMACEUTICAL CORP., Seoul (KR)

(72) Inventors: Shin Jung PARK, Yongin-si (KR); Yunyoung LIM, Yongin-si (KR); Hyo Jun KIM, Yongin-si (KR); Bo Hee HONG, Yongin-si (KR); Yun Sik KIM, Yongin-si (KR)

(21) Appl. No.: 18/039,958

(22) PCT Filed: Dec. 23, 2021
(86) PCT No.: PCT/KR2021/019777

§ 371 Date: Jun. 1, 2023

(30) Foreign Application Priority Data
Dec. 24, 2020 (KR) 10-2020-0183904

(51) Int. Cl. A61K 36/54; A61K 9/00; A61K 31/11; A61K 31/12; A61K 31/192; A61P 1/04

(52) U.S. Cl. CPC A61K 36/54; A61K 9/0056; A61K 31/11; A61K 31/12; A61K 31/192; A61P 1/04; A61K 2236/331; A61K 2236/39; A61K 2236/53

(57) ABSTRACT

The present invention is a pharmaceutical or food composition comprising a pretreated extract and an isolate and an isolated and purified product of Cinnamomum cassia showing a pharmacological effect. It relates to an active ingredient showing physiological activity in the Cinnamomum cassia extract.

### TECHNICAL FIELD

The present invention relates to a composition capable of preventing, improving or treating gastritis or peptic ulcer such as gastric ulcer, which shows an improved pharmacological or food effect comprising a Cinnamomum cassia extract, a fraction of the extract, an isolate of the fraction or a compound isolated and purified therefrom.

### BACKGROUND ART

Stomach is a part of the digestive tract, and is a bulge-like part like a pocket between the esophagus and small intestine (duodenum), and is an organ that stores food that enters through the esophagus, crushes it for easy digestion, and regulates the delivery of food to the duodenum, thereby harmonizing with secretion of digestive enzymes, for efficient digestion and absorption. Factors that adversely affect human gastrointestinal function are extremely diverse in nature and can occur in the upper gastrointestinal tract, lower gastrointestinal tract or both parts, and there are a wide range of gastrointestinal disorder factors including genetic, physiological, environmental and psychological factors. Representative diseases of the upper gastrointestinal tract include gastritis, and peptic ulcer which is a common name of gastric ulcer and duodenal ulcer. Gastritis refers to damage and inflammation of the gastric mucosa, and gastric ulcer refers to when such damage penetrates the mucous membrane and invades the submucosal tissue and muscle layer. In addition, duodenal ulcer is an ulcer that occurs in the duodenum, and gastric ulcer and duodenal ulcer are collectively called peptic ulcer. These gastritis and peptic ulcer are known to be caused by an imbalance of gastric acid, anti-inflammatory drugs and bacterial infection called attack factors, and mucus, cell regeneration and alkali secretion, and the like, called defense factors.

Treatment methods of gastritis and peptic ulcer mainly include antacids that neutralize excessively secreted gastric acid, histamine antagonists for the purpose of inhibiting acid secretion, proton pump inhibitors, cholinergic inhibitors, and gastric mucosal protective agents that increase the resistance of the gastric internal membrane to digestive juices and aid recovery, and the like, and recently, there is a drug treatment method that prescribes a combination of the above drugs and antibiotics to remove Helicobacter pylori, and the like. The characteristics of antacids are that they are fast-acting, and they neutralize gastric acid by raising the pH in the stomach, thereby protecting the gastric mucosa from damage caused by gastric acid. However, administration of inorganic substances may affect the smooth muscle of the gastrointestinal tract, causing constipation, diarrhea, or allergic rejection.

As a histamine receptor blocking secretion inhibitor widely used in peptic ulcer, cimetidine is representative, and also, there are ranitidine, famotidine, roxatidine, and the like as its derivatives, and they block histamine receptors in the gastric mucosa and histamine molecules act to prevent gastric cells from secreting acid. Although they show an excellent anti-ulcer effect in clinical practice, there is a disadvantage in that the regenerated mucosa and submucosal tissue treated have a weaker structure than normal tissue, and therefore, it is easily damaged by attack factors such as gastric acid again after stopping drug administration, and thus the recurrence rate is high. In addition, ranitidine has a disadvantage that the gastric mucosa protection ability is not good because it is not effective in disease such as acute gastritis caused by ethanol, and also, recently, according to the U.S. Food and Drug Administration, the nitrite and dimethylamine groups contained in ranitidine are decomposed and combined by themselves over time to produce NMDA (a carcinogenic substance called N-nitrosodimethyl), and therefore, it is no longer used.

Proton pump inhibitors which have been developed relatively recently include omeprazole and lansoprazole, and the like, and are known to have a strong acid secretion inhibitory effect by inhibiting acid secretion in gastric parietal cells at the final stage, but the recurrence rate is high and side effects such as diarrhea, fever, headache, fatigue, and the like have been reported.

In case of gastric mucosal protective agents, long-term treatment is generally required and there are disadvantages that the dosage is large, but unlike aggressor inhibitors, the regenerated mucosa is known to recover similarly to normal.

Helicobacter pylori, a type of bacteria that inhabit the stomach, is known as a representative cause of peptic ulcer recurrence. Helicobacter pylori is a gram-negative bacillus that inhabits the junction between epithelial cells of the gastric mucosa and causes chronic gastric ulcer, and the treatment to eradicate it has been carried out and many achievements have been made so far, but it has problems such as efficacy, side effects, and the appearance of resistant strains, and a safe and reliable eradication method has not been established.

### DISCLOSURE

### Technical Problem

Accordingly, the present inventors have made intensive efforts to find a substance useful for prevention, improvement or treatment of gastritis and peptic ulcer, which exhibits excellently improved pharmacological effects without causing side effects to the human body, and as a result, they have confirmed that a Cinnamomum cassia extract, a fraction of the extract, an isolate of the fraction or compounds isolated and purified therefrom can be usefully used for prevention, improvement or treatment of gastritis and peptic ulcer.

Thus, it is an object of the present invention is to provide a pharmaceutical or food composition for prevention, improvement or treatment of gastritis or peptic ulcer comprising a Cinnamomum cassia extract, a fraction of the extract, an isolate of the fraction, an active ingredient isolated therefrom or its pharmaceutically acceptable salt as an active ingredient.

### Technical Solution

To achieve the above object, the present invention provides a pharmaceutical or food composition for prevention, improvement or treatment of gastritis or peptic ulcer comprising a Cinnamomum cassia extract, a fraction of the extract, an isolate of the fraction, an active ingredient isolated therefrom or pharmaceutically acceptable salt thereof as an active ingredient.

As one example, the Cinnamomum cassia extract, fraction of the extract or isolate of the fraction may comprise one or more kinds of isolated active ingredients selected from the group consisting of compounds represented by Chemical formulas 1 to 6 below.

As one example, the isolated active ingredients may be selected from the group consisting of compounds represented by Chemical formulas 1 to 6 below.

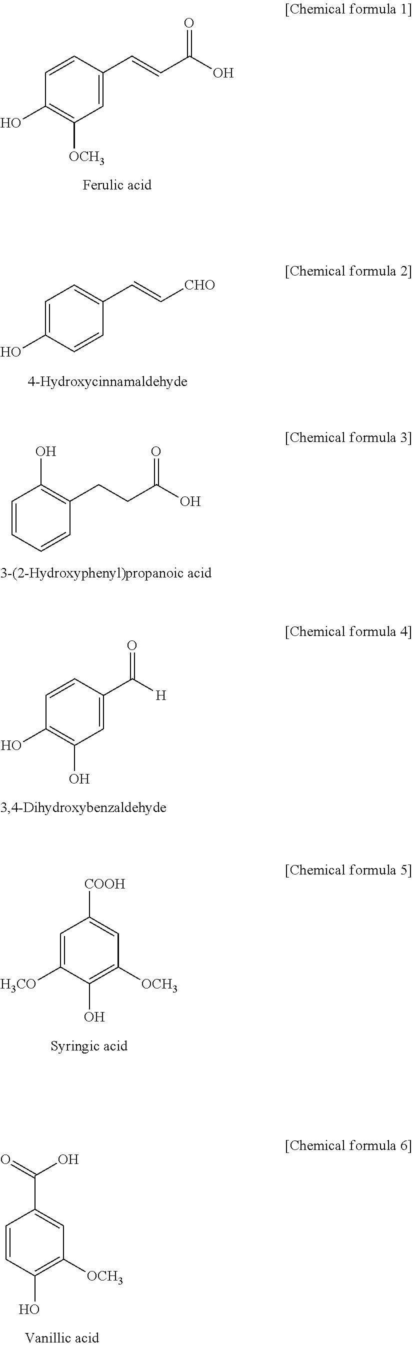 [SMILES: COc1cc(/C=C/C(=O)O)ccc1O ; O=C/C=C/c1ccc(O)cc1 ; O=C(O)CCc1ccccc1O ; O=Cc1ccc(O)c(O)c1 ; COc1cc(C(=O)O)cc(OC)c1O ; COc1cc(C(=O)O)ccc1O]

### Advantageous Effects

The Cinnamomum cassia extract, fraction of the extract or isolate of the fraction, and active ingredients of Chemical formulas 1 to 6 isolated and purified therefrom according to the present invention have excellent anti-inflammation and gastric ulcer inhibitory effects, and thus, they are very useful as an agent for prevention, improvement or treatment of gastritis or peptic ulcer, or a functional food.

### DESCRIPTION OF DRAWINGS

FIG. 1 is a graph which shows HPLC data of the Cinnamomum cassia extract comprised in the composition according to the present invention.

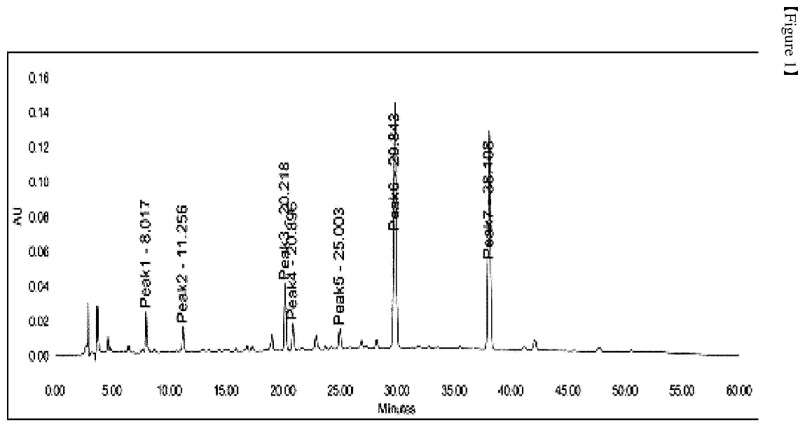

FIG. 2 is a graph which shows the NO inhibition rate of the Cinnamomum cassia solvent fraction comprised in the composition according to the present invention by type.

FIG. 3 is a graph which shows the NO inhibition rate of the isolate comprised in the composition according to the present invention by type (N1 to N6).

FIG. 4 is a graph which shows the NO inhibition rate of the isolate comprised in the composition according to the present invention by type (N2-1 to N2-6).

FIG. 5 is a graph which shows the NO inhibition rate of the isolate comprised in the composition according to the present invention by type (R2-1 to R2-8).

FIG. 6 is a graph schematizing the method for fractioning and isolating and purifying the Cinnamomum cassia extract according to the present invention.

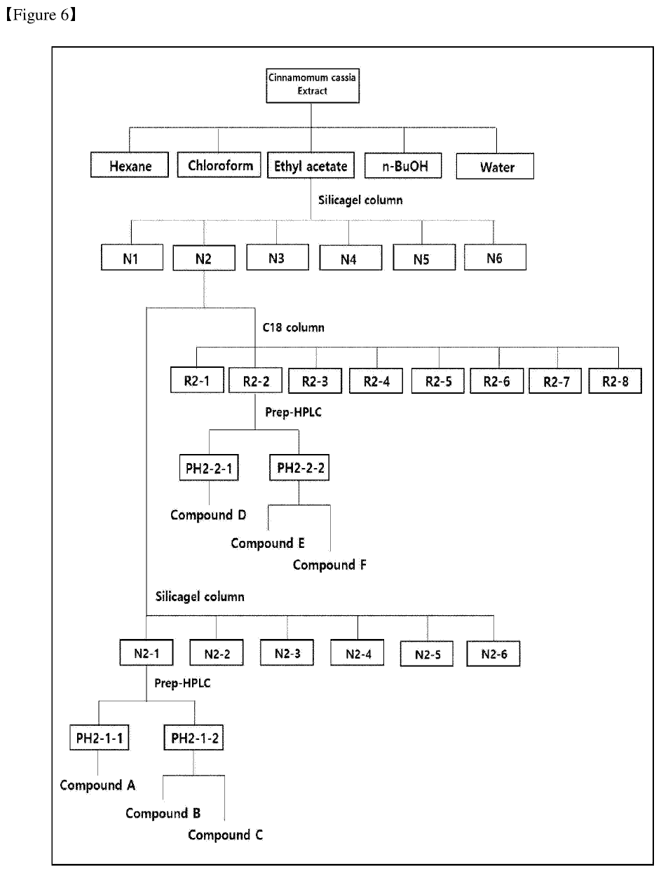

FIG. 7 is a graph of comparing the gastric ulcer inhibition rate of the Cinnamomum cassia extract, fraction and active ingredients, ferulic acid, p-cumaraldehyde, and 3,4-dihydroxybenzaldehyde comprised in the composition according to the present invention with an Artemisia asiatica extract and rebamipide.

### BEST MODE

With this, the present invention will be described in detail.

As one aspect, the present invention relates to a pharmaceutical or food composition for prevention, improvement or treatment of gastritis or peptic ulcer comprising a Cinnamomum cassia extract, a fraction of the extract, an isolate of the fraction, an active ingredient isolated therefrom or pharmaceutically acceptable salt thereof as an active ingredient.

In the present invention, ‘Cinnamomum cassia’ is an evergreen broad-leaved arboreous tree of Ranunculus japonicus order, Cinnamomum camphora family, belonging to Dicotyledonous plants, and its origin is China, and is distributed in Sri Lanka, Indochina and Korea (Jeju) and refers to branches or bark of the Cinnamomum cassia tree (cinnamon tree) that grows up to about 8 m in the mountains.

In the present invention, the term “Cinnamomum cassia extract” means an extract obtained by extracting Cinnamomum cassia. As one specific example, the Cinnamomum cassia extract may be prepared by drying Cinnamomum cassia according to a conventionally known method or by cutting or pulverizing it in an appropriate size for extraction and extracting it using an appropriate extraction solvent, and before extraction, pretreatment may be passed through.

As an extraction method, extraction may be performed using a known herb medicine extraction method such as hot water extraction, cool water extraction, reflux cooling extraction or ultrasonic extraction, or the like, but not limited thereto. In addition, the extract may comprise all of the extraction solution itself, as well as, diluted solution or concentrated solution of the extraction solution, and a dried matter obtained by drying extraction solution.

As one aspect, the Cinnamomum cassia extract of the present invention may be an extract that passes through pretreatment before extracting Cinnamomum cassia with a polar solvent (namely, a polar solvent extract of pretreated Cinnamomum cassia).

As one aspect, the pretreatment may be treating Cinnamomum cassia with a non-polar solvent.

As one specific example, the non-polar solvent may be ethyl acetate.

The non-polar solvent may be used in a volume of 0.5 times to 5 times, 0.7 times to 4 times, or 1 time to 3 times, based on the Cinnamomum cassia weight (preferably, dry weight), but not limited thereto.

As one specific example, pretreatment may be performed by immersing and stirring the cut or pulverized Cinnamomum cassia in a non-polar solvent such as ethyl acetate at a temperature of 20 to 35° C., or room temperature, for time of about 10 minutes to 5 hours, 20 minutes to 4 hours, or 30 minutes to 3 hours.

In one aspect, after completing the pretreatment, Cinnamomum cassia may be washed before extracting it with a polar solvent.

Furthermore, the Cinnamomum cassia extract according to the present invention may be a polar solvent, preferably, water extract of pretreated Cinnamomum cassia as above.

The polar solvent used for extraction may be used in a volume of 5 times to 12 times, 6 times to 10 times or 8 times, based on the Cinnamomum cassia weight (preferably, dry weight), but not limited thereto.

As one specific example, the Cinnamomum cassia pretreated with the non-polar solvent may be extracted at a temperature of 70 to 100° C., or 80 to 100° C. for 1 hour to 7 hours, 2 hours to 6 hours or 5 hours. The extraction may be performed once to several times, once to 3 times, or once to twice, and the extracted pretreated extract may be further filtered, concentrated and/or dried, and then, the used method may be filtering, concentrating and frying methods commonly used for preparation of extracts without limitation.

In one embodiment according to the present invention, Cinnamomum cassia was dried and minced, and ethyl acetate in a volume of 2 times of Cinnamomum cassia was added and immersed and stirred at a room temperature for 1 hour, and after removing ethyl acetate, the pretreated Cinnamomum cassia herb medicine was washed with water, and thus water corresponding to 8 times of the Cinnamomum cassia herb medicine was added and extraction was performed at about 90° C. for 5 hours, and this was repeated twice. The obtained extract was filtered, concentrated under reduced pressure, vacuum dried or spray dried to prepare a Cinnamomum cassia ethyl acetate pretreated water extract.

In the present invention, the term “fraction” refers to a result obtained by a fractionation method for separating a specific component or a specific group from a mixture comprising various components. The fraction of the Cinnamomum cassia extract of the present invention may obtain a polar solvent fraction and a non-polar solvent fraction, respectively, by fractionizing the Cinnamomum cassia extract using a polar solvent such as water, methanol, ethanol, butanol or a non-polar solvent such as hexane, ethyl acetate, chloroform, as a fractionizing solvent. As one specific example, the fraction of the Cinnamomum cassia extract may be an ethyl acetate fraction of the Cinnamomum cassia extract.

In one aspect, as the amount of the fractionizing solvent used, by adding a polar or non-polar solvent such as water, carbon number 1 (C1) to 4 (C4) alcohol, chloroform, ethyl acetate, hexane, butanol or mixed solvent thereof in a volume of 1 to 5 times, 1 to 10 times or 1 to 20 times, based on the Cinnamomum cassia extract 1 kg, a polar or non-poler solvent soluble layer may be obtained by extraction and separation over once to 10 times, preferably, twice to 5 times. In addition, this solvent fractionization may be made sequentially.

In one example of the present invention, the water extract (1 Kg) of Cinnamomum cassia pretreated with ethyl acetate, obtained above was solvent fractionized with H₂O/n-hexane, H₂O/chloroform, H₂O/ethyl acetate, H₂O/n-butanol, by 800 ml each, sequentially, and then the solvent was removed with a concentrator and a vacuum drier, to prepare a Cinnamomum cassia solvent fraction (n-hexane, chloroform, ethyl acetate, n-butanol fractions).

In one example, the fraction of the Cinnamomum cassia extract shows the NO inhibition ratio of 4% or more, 7% or more, 19% or more, 50% or more, or 60% or more. In a preferable aspect, the fraction shows the NO inhibition ratio of 60% to 70%, 60 to 80% or 65% to 85%.

In the present invention, the term “isolate” refers to one obtained by further performing a common separating process to the fraction.

As one example, this isolate may be an isolate obtained by various purification methods conducted additionally such as passing a fraction of the Cinnamomum cassia extract according to the present invention through an ultrafiltration membrane having a constant molecular weight cut-off value, or various chromatography (produced for separation according to size, charge, hydrophobicity or affinity).

In one aspect, this isolate may be obtained by isolating a fraction with higher physiological activity than the fraction, and it is also called an active fraction or effective fraction.

In one aspect, the chromatography may be column chromatography, thin layer chromatography (TLC) or high performance liquid chromatography (HPLC), but not limited thereto, and it may be performed using various chromatography known in the art. The column chromatography may separate and purify compounds by performing column chromatography using a filler selected from the group consisting of silica gel, Sephadex, LH-20, ODS gel, C-18(RP-18), Toyopearl and XAD resin, and the column chromatography may be performed several times by selecting an appropriate filler, if necessary, but not limited thereto. For using the chromatography, elution solvent, elution rate and elution time may be applied to the solvent, rate or time generally used in the art.

In one example of the present invention, the ethyl acetate solvent fraction obtained by fractionizing the Cinnamomum cassia extract with ethyl acetate was eluted using silica gel (normal phase) and chloroform:methanol as an elution solvent, and them the isolated isolate was subject to concentration drying.

In one example, the isolate of the Cinnamomum cassia extract exhibits the NO inhibition rate of 4% or more, 7% or more, 15% or more, 20% or more, 50% or more or 60% or more. In a preferable aspect, the fraction exhibits the NO inhibition rate of 60% to 100%, 70 to 100% or 75% to 100%.

In addition, a specific active ingredient may be isolated by separation and purification by the aforementioned chromatography from the Cinnamomum cassia isolate. The separated and purified active ingredient refers to a single substance showing physiological activity.

In one example, the separated and purified active ingredient according to the present invention may be obtained using the chromatography and/or Prep-HPLC (preparative high-performance liquid chromatography) for the isolate. Then, as a column for HPLC, a C-18 reversed-phase column may be used, and as an elution solvent, acetonitrile and water may be used, but not limited thereto.

In one example, the separated and purified active ingredient according to the present invention may be selected from the group consisting of compounds represented by Chemical formulas 1 to 6 below.

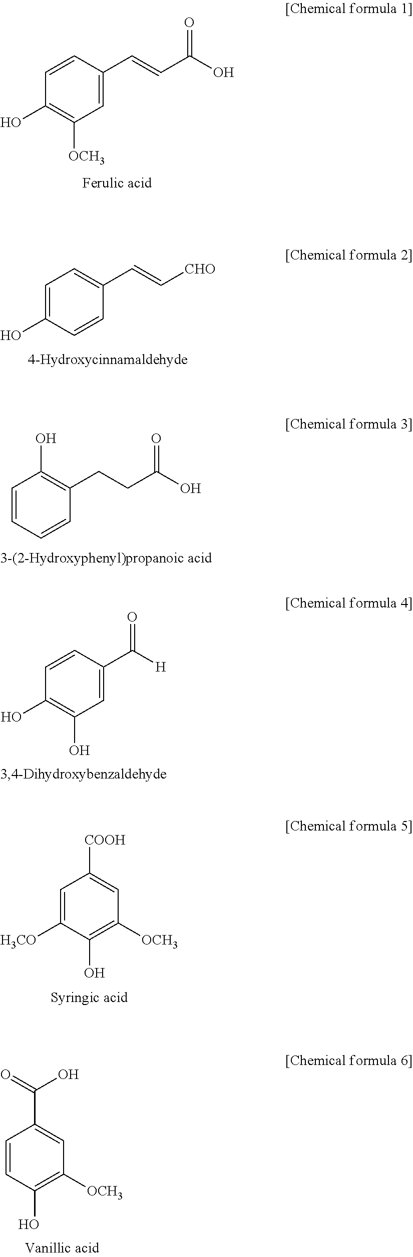 [SMILES: COc1cc(/C=C/C(=O)O)ccc1O ; O=C/C=C/c1ccc(O)cc1 ; O=C(O)CCc1ccccc1O ; O=Cc1ccc(O)c(O)c1 ; COc1cc(C(=O)O)cc(OC)c1O ; COc1cc(C(=O)O)ccc1O]

The Cinnamomum cassia extract, fraction of the extract or isolate of the fraction comprised in the composition according to the present invention may comprise 1 kind or more, 2 kinds or more, 3 kinds or more, 4 kinds or more, 5 kinds or more, 6 kinds, 1 kind to 2 kinds, 1 kind to 3 kinds, 1 kind to 4 kinds, 1 kind to 5 kinds, 1 kind to 6 kinds, 2 kinds to 3 kinds, 2 kinds to 4 kinds, 2 kinds to 5 kinds, 2 kinds to 6 kinds, 3 kinds to 4 kinds, 3 kinds to 5 kinds, 3 kinds to 6 kinds, 4 kinds to 5 kinds, 4 kinds to 6 kinds, or 5 kinds to 6 kinds selected from the group consisting of compounds represented by the Chemical formulas 1 to 6.

In addition, the composition of the present invention may comprise a pharmaceutically acceptable slat of the separated and purified active ingredient. As the salt, an acid addition salt formed by a pharmaceutically acceptable free acid is useful. The term of the present invention, “pharmaceutically acceptable salt” refers to any concentration of a compound having an effective action that is relatively non-toxic and harmless to a patient, which is any organic or inorganic addition salt of the compound in which side effects attributable to the salt do not reduce beneficial efficacy of the compound according to the present invention.

The acid addition salt is prepared by a common method, for example, by dissolving a compound in an excessive amount of acid aqueous solution, and precipitating this salt using a water-miscible organic solvent, for example, methanol, ethanol, acetone or acetonitrile. An equal molar amount of compound and acid or alcohol (e.g., glycol monomethylether) in water were heated, and then the mixture was evaporated to dryness, or the precipitated slat may be suction filtered.

Then, as the free acid, an organic acid and an inorganic acid may be used, and as the inorganic acid, chloric acid, phosphoric acid, sulfuric acid, nitric acid, tartaric acid, and the like may be used, and as the organic acid, methane sulfonic acid, p-toluene sulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like may be used, but not limited thereto.

Furthermore, using a base, a pharmaceutically acceptable metal salt may be prepared. The alkali metal salt or alkali earth metal salt is obtained, for example, by dissolving a compound in an excessive amount of alkali metal hydroxide or alkaline earth metal hydroxide solution, and filtering an undissolved compound salt, and then evaporating and drying the filtrate. Then, as the metal salt, in particular, it is pharmaceutically suitable to prepare a sodium, potassium or calcium salt as the metal salt, but not limited thereto. In addition, the corresponding silver salt may be obtained by reacting an alkali metal or alkali earth metal salt with a suitable silver salt (e.g., silver nitrate).

The pharmaceutically acceptable slat of the compound of the present invention, unless otherwise indicated, comprises a salt of acidic or basic groups which may be present in the compounds described in Chemical formulas 1 to 6 above.

The composition of the present invention comprises an active ingredient itself selected from the group consisting of Chemical formulas 1 to 6 as above, or a Cinnamomum cassia extract comprising this active ingredient, a fraction of the extract, or an isolate of the fraction, thereby exhibiting an excellent effect on prevention, improvement or treatment of gastritis, or peptic ulcer such as gastric ulcer and duodenal ulcer.

In one example, the composition of the present invention may be a pharmaceutical composition.

The pharmaceutical composition of the present invention may comprise the Cinnamomum cassia extract, fraction of the extract or isolate of the fraction in an amount of 10 to 90% by weight, based on the total weight of the composition.

On the other hand, the pharmaceutical composition may comprise one or more kinds selected from the group consisting of ferulic acid, 4-hydroxycinnamaldehyde, 3-(2-hydroxyphenyl)propanoic acid, 3,4-dihydroxybenzaldehyde, syringic acid and vanillic acid as an active ingredient, represented by Chemical formulas 1 to 6, respectively.

The content of the extract, fraction, isolate or active ingredient in the composition may be increased or reduced according to the needs of the type of preparation, administration route, and the like, when the composition is formulated.

The pharmaceutical composition of the present invention may be administered orally or parenterally, and may be used in a general pharmaceutical preparation form. A preferable pharmaceutical preparation includes preparations for oral administration such as tablet, pill, powder, granule, hard or soft capsule, liquid and suspension, and these pharmaceutical preparations may be prepared using a pharmaceutically acceptable common carrier, for example, an excipient, binder, disintegrating agent, glydent, solubilizer, suspending agent, preservative or extender, or the like, in case of preparations for oral administration.

The dose of the pharmaceutical composition of the present invention may be determined by an expert according to various factors such as the patient's condition, age, body weight, and disease progression level, and the like, but in general, as the extract, 9.7˜2,919 mg per day may be administered once or in several divided doses, and preferably, 29.2˜2,919 mg, more preferably, 68.1˜2,919 mg may be administered. However, in case of long-term ingestion, the amount may be less than the above range, and the active ingredient may be used in an amount over the above range because there is no problem in terms of safety.

Moreover, the composition of the present invention may be a food composition. The food is a health supplement food, a health functional food, a functional food, or the like, but not limited thereto, and includes those which the Cinnamomum cassia pretreated extract of the present invention is added to natural food, processed food, or general food materials, or the like. Herein, the term functional refers to obtaining a useful effect for health purposes such as regulating nutrients or physiological effects on the structure and function of the human body.

The health supplement food, health functional food or functional food of the present invention may be prepared by commonly used methods in the art, and during preparation, they may be prepared by adding a raw material and a component commonly added in the art. In addition, different from general medicines, there are no side effects that may occur when taking medicines for a long period of time by using the currently consumed herbal medicine as a raw material, and they are excellent in portability, and therefore, the health functional food of the present invention is possible to be ingested as an adjuvant for enhancing an effect for preventing or improving gastritis or gastric ulcer. The mixed amount of the active ingredient may be appropriately determined according to the purpose of use (prevention, health or therapeutic treatment). In general, when preparing food, the fraction according to the present invention or an isolate of the fraction may be comprised in an amount of 10 to 90% by weight.

On the other hand, the food composition may comprise one or more kinds selected from the group consisting of ferulic acid, 4-hydroxycinnamaldehyde, 3-(2-hydroxyphenyl)propanoic acid, 3,4-dihydroxybenzaldehyde, syringic acid and vanillic acid as an active ingredient, represented by Chemical formulas 1 to 6, respectively.

The content of the extract, fraction, isolate or active ingredient in the composition may be increased or reduced according to the needs of the type of food, administration route, and the like, when the composition is prepared as food.

The effective dose may be used according to the effective dose of the pharmaceutical composition, but in case of long-term ingestion for improvement or maintenance of gastritis or gastric ulcer, it may be under the above range, and as Cinnamomum cassia is used as food conventionally, there is no problem in terms of safety, and therefore, it may be used in an amount over the above range.

There is no particular limitation on the type of food. The food composition comprising an active ingredient selected from the group consisting of the Cinnamomum cassia extract, fraction of the extract or isolate of the extract, or Chemical formulas 1 to 6 may be used in a form of preparations for oral administration such as tablet, pill, powder, granule, hard or soft capsule, liquid and suspension, and these preparations may further comprise an acceptable common food supplement additive. The term “food supplement additive” is added to the manufacture of health functional food of each formulation, and those skilled in the art may appropriately select and use it. Examples of the food supplement additive include various nutrients, vitamins, minerals (electrolytes), flavoring agents such as synthetic flavoring agents and natural flavoring agents, coloring agents an fillers, pectic acid and its salts, alginic acid and its salts, organic acids, protective colloidal thickeners, pH adjusting agents, stabilizers, preservatives, glycerin, alcohol, carbonating agents used in carbonated beverages, and the like, but the type of the food supplement additive of the present invention is not limited by the above examples.

In addition, the food composition of the present invention may be used by adding the composition as it is, or together with other food or food compositions, and may be appropriately used according to a common method. The mixed amount of the active ingredients may be suitably determined according to the purpose of its use (prevention, improvement or therapeutic treatment).

Examples of the food include meat, sausage, bread, chocolate, candy, snacks, confectionery, pizza, ramen, other noodles, gums, dairy products including ice cream, various soups, beverages, tea, drinks, alcoholic beverages and vitamin complexes, and other nutritional supplements, but are not limited to these types of food.

According to one example of the present invention, the Cinnamomum cassia extract according to the present invention, fraction of the extract or isolate of the fraction, and active ingredients of Chemical formulas 1 to 6 separated and purified therefrom reduce the NO production amount, and inhibit PGE2, and thus exhibit an excellent anti-inflammatory effect, and also exhibit an excellent gastric ulcer inhibitory effect.

### MODE FOR INVENTION

Hereinafter, the present invention will be described in more detail by examples. These examples are intended to illustrate the present invention only, and therefore, it should not be construed that the scope of the present invention is limited by these examples.

### Example 1. Preparation of Extract and Solvent Fraction According to the Present Invention

1) Preparation of Cinnamomum cassia Pretreated Extract

The Cinnamomum cassia pretreated extract according to the present invention was prepared. Specifically, 2 times of ethyl acetate was added to Cinnamomum cassia herb medicine, and it was immersed and stirred at a room temperature for 1 hour or more. Ethyl acetate was removed and the Cinnamomum cassia herb medicine was washed with water, and then 8 times water was added and it was extracted at about 90° C. for 5 hours (repeat twice). The extract was filtered, concentrated under reduced pressure, vacuum dried or spray dried to prepare a Cinnamomum cassia ethyl acetate pretreated water extract (extract obtained: 16˜26→1).

2) Preparation of Cinnamomum cassia Solvent Fraction

The Cinnamomum cassia solvent fraction according to the present invention was prepared as follows. Specifically, the Cinnamomum cassia pretreated extract 1 KG of Example 1 was under solvent fractionization with 800 ml each of H₂O/n-Hexane, H₂O/Chloroform, H₂O/Ethyl acetate, H₂O/n-Butanol, sequentially, and then the solvent was removed with a concentrator and a vacuum dryer to prepare a Cinnamomum cassia solvent fraction.

TABLE 1
Yield of Cinnamomum cassia solvent fraction

| Solvent | Obtained (g) | Yield (%) | Remark |
|---|---|---|---|
| n-Hexane | 1.3 | 0.1 | — |
| Chloroform | 2.6 | 0.3 | — |
| Ethyl acetate | 10.2 | 1.0 | Active |
| n-Butanol | 52.1 | 5.1 | — |

### Example 2. Separation and Purification of Biologically Active Substance According to the Present Invention 1

1) Separation of Biologically Active Substance Through Silica Gel Column Chromatography

The biologically active substance through silica gel column chromatography according to the present invention was separated as follows. Specifically, the ethyl acetate solvent fraction among Cinnamomum cassia solvent fractions of Example 1-2 and silica gel (normal phase) as resin used for an open column were used. The ethyl acetate solvent fraction was separated by an absorptive property after filling the silica gel column. The elution solvent was Chloroform:Methanol=5:1, and it was eluted and then the separation level of spots was confirmed on TLC and it was concentrated and dried.

TABLE 2
Yield of silica gel column chromatography isolate

| Isolate | Obtained (mg) | Yield (%) | Remark |
|---|---|---|---|
| N1 | 600 | 6.28 | — |
| N2 | 870 | 9.10 | Active |
| N3 | 1,270 | 13.28 | — |
| N4 | 3,020 | 31.59 | — |
| N5 | 1,450 | 15.17 | — |
| N6 | 2,350 | 24.58 | — |

*N = Normal phase

2) Separation of Biologically Active Substance Through Silica Gel Column Chromatography

The biologically active substance through silica gel column chromatography according to the present invention was separated as follows. Specifically, the isolate N2 in the column chromatography of Example 2-1 and silica gel (normal phase) as resin used for an open column were used, and the isolate N2 was separated by an absorptive property after filling the silica gel column. The elution solvent was Hexane:Ethyl acetate=1:3, and it was eluted and then the separation level of spots was confirmed on TLC and it was concentrated and dried.

TABLE 3
Yield of silica gel column chromatography isolate

| Isolate | Obtained (mg) | Yield (%) | Remark |
|---|---|---|---|
| N2-1 | 315 | 43.2 | Active |
| N2-2 | 194 | 26.6 | — |
| N2-3 | 45 | 6.2 | — |
| N2-4 | 39 | 5.4 | — |
| N2-5 | 27 | 3.6 | — |
| N2-6 | 109 | 15.0 | — |

*N = Normal phase

3) Purification of Biologically Active Single Substance Through Prep-HPLC

The biologically active substance through Prep-HPLC according to the present invention was separated as follows. Specifically, the isolate N2-1 in the silica gel column chromatography of Example 2-2 and Prep-HPLC equipped with a C18 column were used, and the N2-1 was separated into PH2-1-1 and PH2-1-2 by a property of the C18 column. The elution solvent was Acetonitrile:H₂O, and it was eluted and then the separation level of spots was confirmed on TLC and it was concentrated and dried. PH2-1-1 was purified into compound A by the property of the C18 column. The elution solvent was Acetonitrile:H₂O, and it was eluted and then the separation level of spots was confirmed on TLC and it was concentrated and dried. PH2-1-2 was purified into compound B and compound C by the property of the C18 column. The elution solvent was Acetonitrile:H₂O, and it was eluted and then the separation level of spots was confirmed on TLC and it was concentrated and dried.

TABLE 4
Yield of Prep-HPLC purified product

| | | | Remark (purified |
|---|---|---|---|
| Isolate | Obtained (mg) | Yield (%) | product) |
| PH2-1-1 | 313.3 | 29.5 | Compound A |
| PH2-1-2 | 181.3 | 17.1 | Compound |
| | | | BCompound C |

*PH = Prep-HPLC

### Example 3. Separation and Purification of Biologically Active Substance According to the Present Invention 2

1) Separation of Biologically Active Substance Through C18 Column Chromatography

The biologically active substance through C18 column chromatography according to the present invention was separated as follows. Specifically, the isolate N2 in the column chromatography of Example 2-1 and C18 (reverse phase) as resin used for an open column were used, and the isolate N2 was separated by an absorptive property after filling the C18 column. The elution solvent was Methanol:H₂O=1:1, and it was eluted, and then the separation level of spots was confirmed on TLC and it was concentrated and dried.

TABLE 5
Yield of C18 column chromatography isolate

| Isolate No | Obtained (mg) | Yield (%) | Remark |
|---|---|---|---|
| R2-1 | 57 | 8.5 | — |
| R2-2 | 79 | 11.7 | Active |
| R2-3 | 76 | 11.3 | — |
| R2-4 | 142 | 21.0 | — |
| R2-5 | 105 | 15.6 | — |
| R2-6 | 106 | 15.8 | — |
| R2-7 | 40 | 5.9 | — |
| R2-8 | 69 | 10.2 | — |

*R = reverse phase

2) Purification of Biologically Active Single Substance Through Prep-HPLC

The biologically active substance through Prep-HPLC according to the present invention was separated as follows. Specifically, the isolate R2-2 in the C18 column chromatography of Example 3-1 and Prep-HPLC equipped with a C18 column were used, and the isolate R2-2 was separated into PH2-2-1 and PH2-2-2 by a property of the C18 column. The elution solvent was Acetonitrile:H₂O, and it was eluted and then the separation level of spots was confirmed on TLC and it was concentrated and dried. PH2-2-1 was purified into compound D by the property of the C18 column. The elution solvent was Acetonitrile:H₂O, and it was eluted and then the separation level of spots was confirmed on TLC and it was concentrated and dried.

TABLE 6
Yield of Prep-HPLC purified product

| | | | Remark (purified |
|---|---|---|---|
| Isolate | Obtained (mg) | Yield (%) | product) |
| PH2-2-1 | 28 | 63.6 | Compound D |
| PH2-2-2 | 16 | 36.3 | Compound E |
| | | | Compound F |

*PH = Prep-HPLC

### Example 4. Identification of Structure by Physical and Chemical Properties of Purified Compounds According to the Present Invention

Measurement of Nuclear Magnetic Resonance (NMR) Spectrum

The NMR spectrum was measured by dissolving 10 mg of pure purified product in each solvent using 1H-NMR (400 MHz) and 13C-NMR (100 MHz).

Mass Spectrum Measurement

After separation and purification, 1 mg of dry powder solid sample was measured by chemical analysis using negative ion FAB-mass spectrum under reduced pressure (10 mmHg). Then, as a measurement solvent, thioglycero was used, and the measurement conditions were that the emitter current was 22˜28 eV and mass spectrometry was performed at 6˜7 KV of accelerated pressure of the ion source.

The result of structural identification of the purified material through the above method is as follows.

1) Compound A

As the result of the structural identification of compound A, it has a molecular structure of C₁₀H₁₀O₄, and the positive FAB-MS molecular weight was measured as 194.18. ¹H-NMR (CD₃OD, 400 MHz) spectrum was δ_{H} 7.57 (1H, d, J=16.0 Hz), 7.17 (1H, d, J=2.0 Hz), 7.05 (1H, dd, J=8.0, 2.0 Hz), 6.80 (1H, d, J=8.0 Hz), 6.30 (1H, d, J=16.0 Hz), 3.88 (3H, s) ppm, and ¹³C-NMR (CD₃OD, 100 MHz) spectrum was δ_{C} 170.2, 149.1, 148.0, 144.2, 128.0, 121.6, 116.6, 114.6, 110.3, 54.4 ppm, and thereby, compound A was identified as ferulic acid.

2) Compound B

As the result of the structural identification of compound B, it has a molecular structure of C₉H₈O₂, and the positive FAB-MS molecular weight was measured as 148.16. ¹H-NMR (CDCl₃, 400 MHz) spectrum was δ_{H} 9.63 (1H, d, J=7.8 Hz), 9.09 (1H, br s), 7.62 (2H, d, J=8.7 Hz), 7.58 (1H, d, J=15.8 Hz), 6.94 (2H, d, J=8.6 Hz), 6.61 (1H, dd, J=15.8, 7.7 Hz) ppm, and the ¹³C-NMR (CDCl₃, 100 MHz) spectrum was δ_{C} 193.9, 161.3, 153.7, 131.6, 127.0, 126.8, 116.8 ppm, and thereby, compound B was identified as 4-hydroxycinnamaldehyde.

3) Compound C

As the result of the structural identification of compound C, it has a molecular structure of C₉H₈O₃, and the positive FAB-MS molecular weight was measured as 164.16. The ¹H-NMR (DMSO-d₆, 400 MHz) spectrum was δ_{H} 7.0 (4H, m), 2.78 (2H, t, J=7.0 Hz), 2.50 (2H, t, J=7.0 Hz) ppm, and the ¹³C-NMR (DMSO-d₆, 100 MHz) spectrum was δ_{C} 174.6, 155.6, 130.1, 127.6, 127.3, 119.3, 115.3, 34.1, 25.9 ppm, and thereby, compound C was identified as 3-(2-hydroxyphenyl)propanoic acid.

4) Compound D

As the result of the structural identification of compound D, it has a molecular structure of C₇H₆O₃, and the positive FAB-MS molecular weight was measured as 138.12. The ¹H-NMR (CD₃OD, 400 MHz) spectrum was δ_{H} 9.68 (1H, s), 7.30 (1H, dd, J=9.48, 1.70 Hz), 7.28 (1H, d, J=1.41 Hz), 6.90 (1H, d, J=8.1 Hz) ppm, and the ¹³C-NMR (CD₃OD, 100 MHz) spectrum was δ_{C} 193.8, 154.5, 148.0, 131.6, 127.2, 117.0, 116.2 ppm, and thereby, compound D was identified as 3,4-dihydroxybenzaldehyde.

5) Compound E

As the result of the structural identification of compound E, it has a molecular structure of C₉H₁₀O₅, and the positive FAB-MS molecular weight was measured as 198.17. The ¹H-NMR (CD₃OD, 400 MHz) spectrum was δ_{H} 7.31 (2H, s), 3.87 (6H, s) ppm, and the ¹³C-NMR (CD₃OD, 100 MHz) spectrum was δ_{C} 167.2, 147.4, 140.1, 120.6, 106.8, 55.9 ppm, and thereby, compound E was identified as syringic acid.

6) Compound F

As the result of the structural identification of compound F, it has a molecular structure of C₈H₈O₄, and the positive FAB-MS molecular weight was measured as 168.15. The ¹H-NMR (CD₃OD, 400 MHz) spectrum was δ_{H} 7.55 (2H, m), 6.84 (d, J=8.8 Hz), 3.89 (3H, s) ppm, and the ¹³C-NMR (CD₃OD, 100 MHz) spectrum was δ_{C} 170.1, 152.6, 148.7, 125.3, 123.2, 115.8, 113.9, 56.4 ppm, and thereby, compound F was identified as vanillic acid.

[Measurement of Anti-Inflammatory Effect in Raw 264.7 Cells]

### Experimental Example 1. Confirmation of Nitric Oxide Production Inhibitory Activity

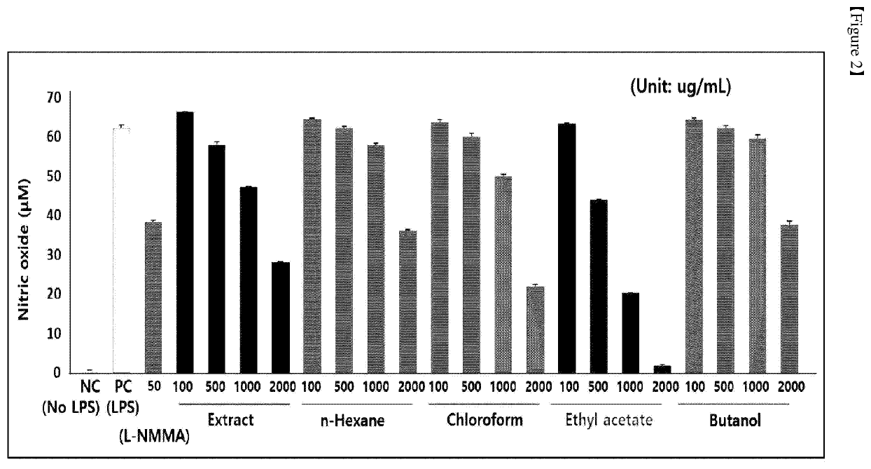

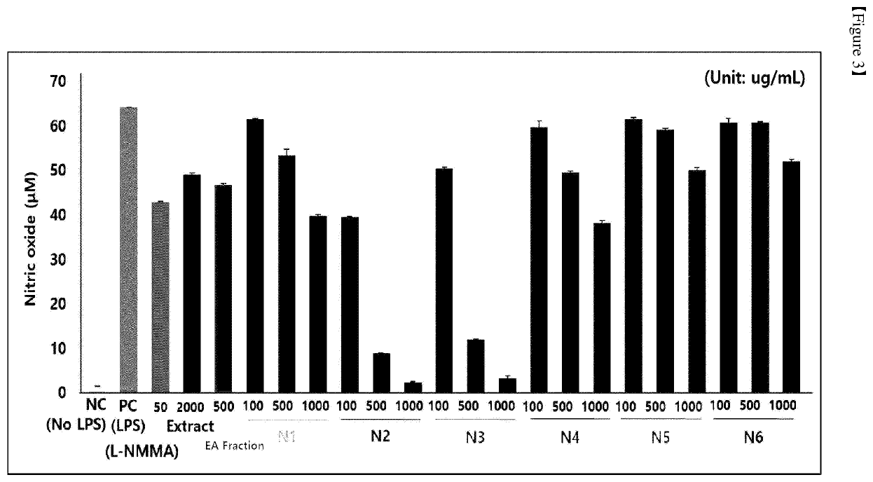

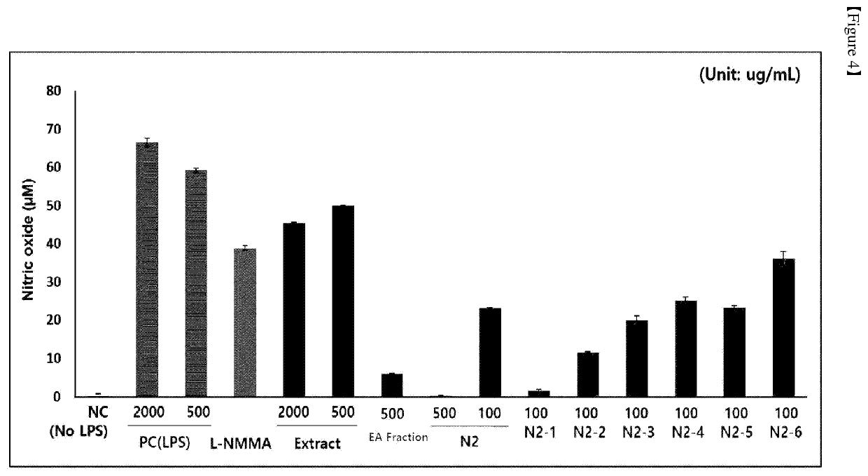

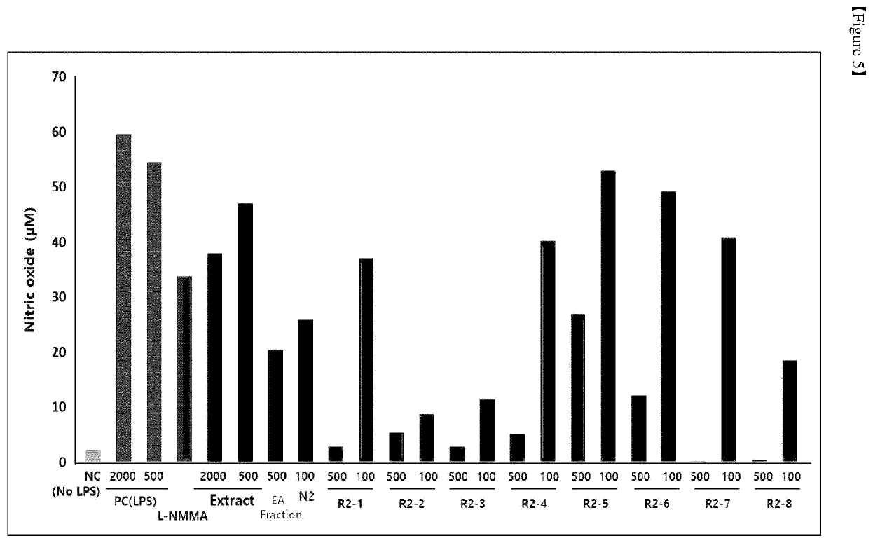

Nitric oxide (NO) measurement was measuring the amount of nitric oxide (NO) in the supernatant of cells as nitrite and nitrate. The safe form after oxidation from nitrite to nitrate was measured using a griess reagent (Sigma, USA). In a 2 well plate, 3×10⁵ cells were cultured for 24 hours. In 24 hours, the solvent fraction, column isolate and Prep-HPLC purified product were treated to each well by concentration. Then, all wells except the normal group were stimulated by adding 100 ng/ml Lipopolysaccharide (LPS). After reacting with the griess reagent for 5 minutes, the absorbance was measured at 540 nm. The calibration curve was prepared using sodium nitrite solution, and the concentration of nitrite was calculated through absorbance. The NO inhibition rate was evaluated by a method of comparing with the group treated only with LPS according to Equation 1 below. The result was shown in the following Tables 7, 8, 9, 10, 11 and 12 and FIGS. 2, 3, 4 and 5.

NO inhibition rate (%)=100−[(NO production amount of sample addition group*×100)/NO production amount of sample non-addition group*] [Equation 1]

*Lipopolysaccharide (LPS) treatment group

TABLE 7
NO inhibition rate of Cinnamomum cassia solvent fraction

| Solvent | NO (μM) | NO inhibition rate (%) | Remark |
|---|---|---|---|
| n-Hexane | 57.87 | 7.1 | — |
| Chloroform | 49.88 | 19.9 | — |
| Ethyl acetate | 20.34 | 67.4 | Active |
| n-Butanol | 59.59 | 4.4 | — |

TABLE 8
NO inhibition of silica gel column chromatography isolate

| Isolate | NO (μM) | NO inhibition rate (%) | Remark |
|---|---|---|---|
| N1 | 53.56 | 16.6 | — |
| N2 | 8.92 | 86.1 | Active |
| N3 | 12.03 | 81.3 | — |
| N4 | 49.53 | 22.8 | — |
| N5 | 59.22 | 7.7 | — |
| N6 | 60.77 | 5.3 | — |

*N = Normal phase

TABLE 9
NO inhibition rate of silica gel column chromatography isolate

| Isolate | NO (μM) | NO inhibition rate (%) | Remark |
|---|---|---|---|
| N2-1 | 1.54 | 97 | Active |
| N2-2 | 11.57 | 80 | — |
| N2-3 | 19.93 | 66 | — |
| N2-4 | 25.21 | 57 | — |
| N2-5 | 23.40 | 61 | — |
| N2-6 | 36.23 | 39 | — |

*N = Normal phase

TABLE 10
NO inhibition rate of Prep-HPLC purified product

| | | | Remark (purified |
|---|---|---|---|
| Isolate | NO (μM) | NO inhibition rate (%) | product) |
| PH2-1-1 | 49.57 | 0 | Compound A |
| PH2-1-2 | 35.18 | 29 | Compound B |
| PH2-1-2′ | 48.54 | 0 | Compound C |

*PH = Prep-HPLC

TABLE 11
NO inhibition rate of C18 column chromatography isolate

| Isolate No | NO (μM) | NO inhibition rate (%) | Remark |
|---|---|---|---|
| R2-1 | 37.13 | 37.8 | — |
| R2-2 | 8.72 | 85.4 | Active |
| R2-3 | 11.38 | 80.9 | — |
| R2-4 | 40.34 | 32.4 | — |
| R2-5 | 53.02 | 11.2 | — |
| R2-6 | 49.15 | 17.7 | — |
| R2-7 | 40.90 | 31.5 | — |
| R2-8 | 18.56 | 68.9 | — |

*R = reverse phase

TABLE 12
NO inhibition rate of Prep-HPLC purified product

| | | | Remark (purified |
|---|---|---|---|
| Isolate | NO (μM) | NO inhibition rate (%) | product) |
| PH2-2-1 | 44.41 | 10 | Compound D |
| PH2-2-2 | 47.83 | 0 | Compound E |
| PH2-2-2′ | 49.88 | 0 | Compound F |

*PH = Prep-HPLC

[Measurement of gastric ulcer inhibitory effect in animals]

### Experimental Example 2. Confirmation of Gastric Ulcer Inhibition Rate

Evaluation of Gastric Ulcer Index in Indomethacin-Induced Animal Model

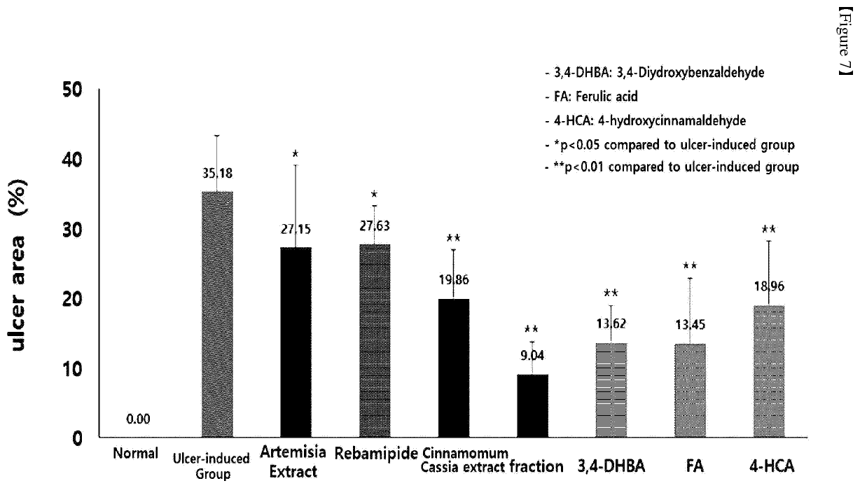

For test animals, 7-week-old male rats without specific pathogens (SPF) were randomly distributed to each group by a randomized method so that the average body weight was distributed as evenly as possible after the adaptation period (7 days). The administration route was single administration of artemisia extract, and rebamipide and ethyl acetate pretreated water extract of Cinnamomum cassia, fraction (N2-1), 3,4-dihydroxybenzaldehyde, ferulic acid, 4-hydroxycinnamaldehyde, as oral administration, respectively, and the dose was calculated and administered as 10 mL/Kg based on the body weight measured on the day of administration. All animals were fasted 48 hours before administration of the test substance and control drug, and then each test substance and control drug were orally administered, and at 30 minutes after administration, indomethacin prepared in advance was orally administered at a dose of 80 mg/kg. After anesthesia with diethyl ether at 5 hours after administration of indomethacin, the stomach was excised and the gastric mucosa was photographed with a digital camera. The area of the damaged part was analyzed using ImageJ software (NIH, Bethesda, MD). The gastric ulcer index was measured according to the following equation. The result was shown in Table 13 and FIG. 7.

Gastric ulcer index (%)=(damaged area/total area)×100 [Equation 2]

TABLE 13

| | | | Inhibition |
|---|---|---|---|
| Group | Test substance | Dose (mg/kg) | rate (%) |
| G1 | — | — | — |
| G2 | — | — | — |
| G3 | Artemisia extract | 20 | 22.8 |
| G4 | Rebamipide | 30 | 21.5 |
| G5 | Cinnamomum cassia extract | 24 | 43.5 |
| G6 | Fraction (N2-1) | 5 | 74.3 |
| G7 | 3,4-Dihydroxybenzaldehyde | 1.5 | 61.3 |
| G8 | Ferulic acid | 1.5 | 61.8 |
| G9 | 4-Hydroxycinnamaldehyde | 2.0 | 46.1 |

As could be seen in FIG. 7 and Table 13, it could be confirmed that the Cinnamomum cassia extract, fraction and 3,4-dihydroxybenzaldehyde, ferulic acid and 4-hydroxycinnamaldehyde separated and purified therefrom exhibited an excellent gastric ulcer inhibition rate, compared to Stillen, which is a conventionally commercially available artemisia extract agent, and Rebamipide, which is a commercially available gastric ulcer therapeutic agent. In particular, 4-dihydroxybenzaldehyde, ferulic acid and p-cumaraldehyde showed a very high level of gastric ulcer inhibition rate despite of the very small amount, and thus, it could be confirmed that an agent comprising them as an active ingredient may be very useful for gastritis and peptic ulcer.

1. A pharmaceutical composition for preventing, improving or treating gastritis or peptic ulcer,
comprising a component selected from the group consisting of a Cinnamomum cassia extract, a fraction of the extract and an isolate of the fraction,
wherein the extract, fraction or isolate comprises one or more kinds of isolated active ingredients selected from the group consisting of compounds represented by Chemical formulas 1 to 6:

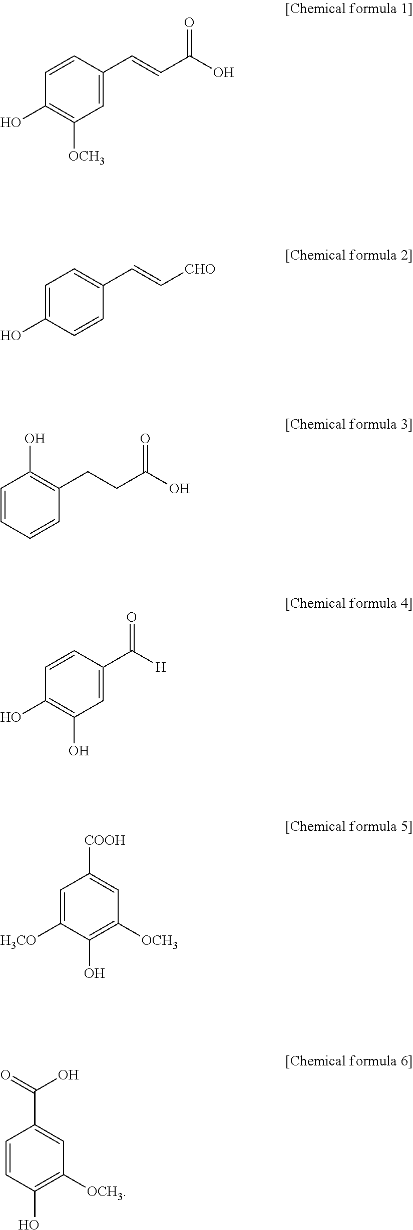 [SMILES: COc1cc(/C=C/C(=O)O)ccc1O ; O=C/C=C/c1ccc(O)cc1 ; O=C(O)CCc1ccccc1O ; O=Cc1ccc(O)c(O)c1 ; COc1cc(C(=O)O)cc(OC)c1O ; COc1cc(C(=O)O)ccc1O]

2. A pharmaceutical composition for preventing, improving or treating gastritis or peptic ulcer, comprising one or more kinds of isolated active ingredients selected from the group consisting of compounds represented by Chemical formulas 1 to 6 or pharmaceutically acceptable salt thereof:

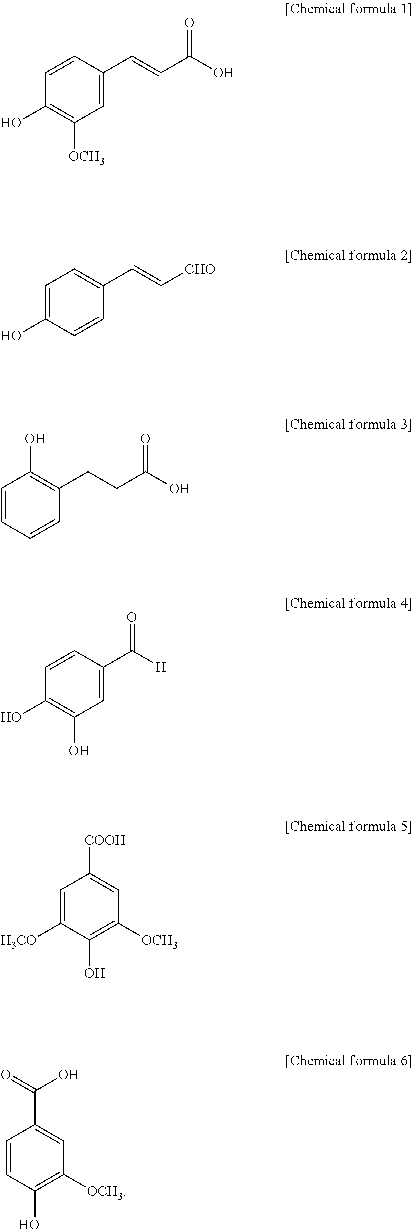 [SMILES: COc1cc(/C=C/C(=O)O)ccc1O ; O=C/C=C/c1ccc(O)cc1 ; O=C(O)CCc1ccccc1O ; O=Cc1ccc(O)c(O)c1 ; COc1cc(C(=O)O)cc(OC)c1O ; COc1cc(C(=O)O)ccc1O]

3. The pharmaceutical composition according to claim 1, wherein the Cinnamomum cassia extract is a polar solvent extract of non-polar solvent pretreated Cinnamomum cassia.

4. The pharmaceutical composition according to claim 3, wherein the non-polar solvent is ethyl acetate, and the polar solvent is water.

5. The pharmaceutical composition according to claim 1, wherein the fraction is a fraction fractionized using a solvent selected from the group consisting of water, methanol, ethanol, butanol, hexane, ethyl acetate and chloroform.

6. The pharmaceutical composition according to claim 5, wherein the fraction of the Cinnamomum cassia extract is an ethyl acetate fraction.

7. The pharmaceutical composition according to claim 1, wherein the isolate is isolated by ultrafiltration membrane or chromatography.

8. The pharmaceutical composition according to claim 7, wherein the chromatography is a silica gel column chromatography or a column chromatography using C-18.

9. The pharmaceutical composition according to claim 1, wherein the one or more kinds of active ingredients selected from the group consisting of Chemical formulas 1 to 6 are obtained by isolating and purifying the isolate by Prep-HPLC.

10. The pharmaceutical composition according to claim 1, further comprising a pharmaceutically acceptable carrier.

11. A food composition for preventing or improving gastritis or peptic ulcer,
comprising a component selected from the group consisting of a Cinnamomum cassia extract, a fraction of the extract and an isolate of the fraction,
wherein the extract, fraction or isolate comprises one or more kinds of isolated active ingredients selected from the group consisting of compounds represented by Chemical formulas 1 to 6:

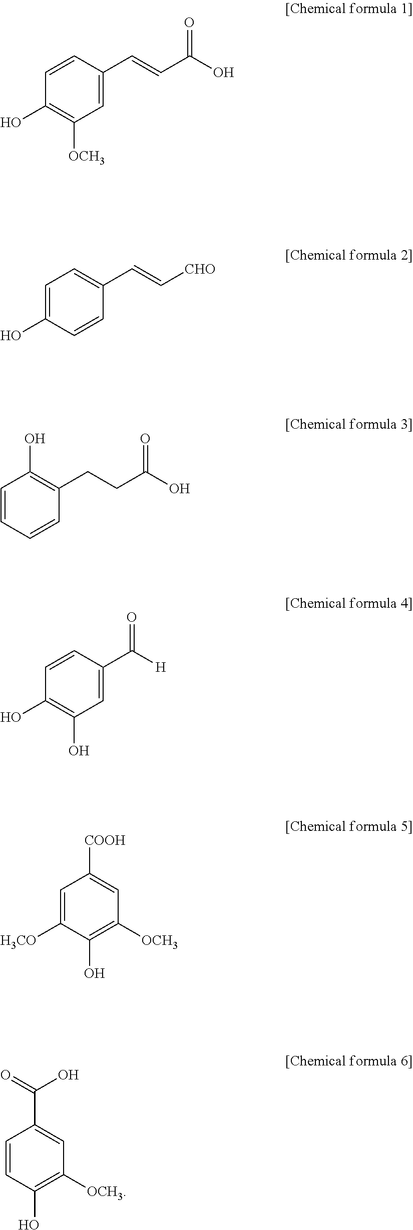 [SMILES: COc1cc(/C=C/C(=O)O)ccc1O ; O=C/C=C/c1ccc(O)cc1 ; O=C(O)CCc1ccccc1O ; O=Cc1ccc(O)c(O)c1 ; COc1cc(C(=O)O)cc(OC)c1O ; COc1cc(C(=O)O)ccc1O]

12. A food composition for preventing or improving gastritis or peptic ulcer, comprising one or more kinds of isolated active ingredients selected from the group consisting of compounds represented by Chemical formulas 1 to 6 or pharmaceutically acceptable salt thereof: 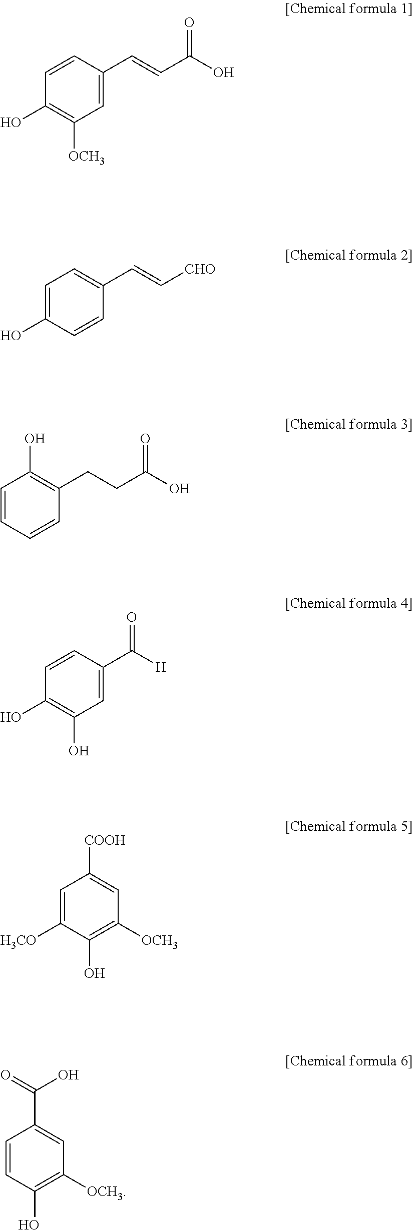 [SMILES: COc1cc(/C=C/C(=O)O)ccc1O ; O=C/C=C/c1ccc(O)cc1 ; O=C(O)CCc1ccccc1O ; O=Cc1ccc(O)c(O)c1 ; COc1cc(C(=O)O)cc(OC)c1O ; COc1cc(C(=O)O)ccc1O]
